(19) United States
(12) Patent Application Publication
(10) Pub. No.: US 2009/0082754 A1
(43) Pub. Date: Mar. 26, 2009

(54) Cannula implantation instrument

(75) Inventors: Axel Hentrich, Hamburg (DE); Burkhard Peters, Wattenbek (DE)

Correspondence Address: BODNER & O'ROURKE, LLP, 425 BROADHOLLOW ROAD, SUITE 108, MELVILLE, NY, 11747, US

(73) Assignee: Ethicon, Inc., Somerville, NJ (US)

(21) Appl. No.: 11/904,196

(22) Filed: Sep. 26, 2007

(51) Int. Cl. A61M 31/00; A61M 25/06

(52) U.S. Cl. 604/506; 604/158

(57) ABSTRACT

A cannula implantation instrument for use with the delivery of guide wire based implants includes a handle having an open slot for receiving a guide wire and a cavity for receiving an implant pre-mounted on the guide wire, a cannula having an open slot for also receiving a guide wire and a sharpened tip for introducing the cannula and guide wire simultaneously into the vas deferens of a patient, and a guide wire on which an intra vas implant is pre-mounted. The instrument further includes a safety clip on the handle that secures the guide wire and/or implant to the handle until released by a physician. The cannula implantation instrument may be ergonomically formed to provide comfort and ease while being handled by the physician.

### BACKGROUND OF THE INVENTION

1. Field of the Invention

This invention relates to implantation cannulas and more specifically relates to implantation cannulas for use with an intra vas implant.

2. Description of the Prior Art

Definitive male contraception is most commonly achieved by a surgical vasectomy. While the vasectomy is an effective contraception method, the procedure is very often painful and has many long term effects that are not always desirable, but are accepted as other methods of definitive contraception are not readily available.

Conventionally, a vasectomy may be performed surgically by the traditional method of removing the vas deferens completely or by cutting and sealing the vas deferens, most commonly referred to as a “keyhole” or “no-scalpel” vasectomy. Patients undergoing either type of vasectomy should consider the procedure permanent and not reversible. In some rare cases, a vasectomy may be reversed by another surgical procedure known as a vasovasostomy; however, the success rate of completely returning the reproductive capabilities of the male with this procedure is poor at best. Post-vasectomy patients are commonly limited in activities for up to four weeks and are often prone to severe and chronic pain lasting years. The vasovasostomy has been an effective means of treating pain arising from vasectomy complications, but as with most microsurgical procedures, is extremely costly. Many males desiring an effective contraception method often hesitate when considering a vasectomy due to the essentially irreversible effects of the surgery and the potential for long lasting pain.

A new method of definitive male contraception utilizing an intra vas implant has been proven to be as effective as vasectomy procedures while having significantly less complications and long term side effects. The intra vas implant (IVI) operates in a manner similar to that of an intra uterine device (IUD) in females. To achieve complete contraception capabilities in males, the IVI is surgically implanted into the vas deferens, blocking the path of sperm from the testicles to the urethra. The conventional method of surgical IVI implantation in most cases is cumbersome and time consuming. An incision is made in the vas deferens utilizing a conventional cannula. A guide wire, having an inner diameter averaging 0.36 millimeters, is placed through the bore of the cannula into the vas deferens. The cannula is then removed and the IVI, having an inner diameter of 4 mm, is beaded onto the guide wire. The beading of the IVI onto the guide wire is particularly frustrating for the clinician due to the minute size of the devices and the significant amount of precision needed. After the IVI is beaded onto the guide wire, it is advanced forward into the vas deferens. The final stage of the implantation includes the removal of the guide wire and treatment of the wound.

Male contraception utilizing an IVI can be permanent or temporary. The device can be removed at any time by making a small incision in the scrotum. Generally, the procedure is non-traumatic in nature, and any side effects are miniscule compared to those of a vasectomy. However, as mentioned previously, due to the size of the devices involved in the procedure, preparation and performing the surgical implantation is time consuming and often frustrating. While the IVI contraception method is effective and promising, a more efficient method of preparing and introducing the implant into the vas deferens is needed to make the surgical procedure more practical.

Various methods of introducing implants, guide wires and catheters into tissue and body anatomy are conventionally available and are described in U.S. Pat. No. 4,306,562 which issued to Thomas A. Osborne, U.S. Pat. No. 4,402,685 which issued to Wolfgang Buhler et al., U.S. Pat. No. 3,677,244 which issued to Robert J. Hassinger, U.S. Pat. No. 3,677,243 which issued to Joseph E. Nerz, U.S. Patent Application Publication No. 2006/0116691 having as a named inventor Fabrice Bonacci, U.S. Patent Application Publication No. 2003/0073934 having as a named inventor David A. Putz and PCT Patent Application Publication No. WO99/22804 having as named inventors Michael J. Licata et al. However, the methods and devices described in the aforementioned patents and published applications, and conventional methods and devices currently in use, are not efficient and practical for use in introducing an IVI into the vas deferens. It is not possible to scale the aforementioned devices to a size that is useable for implanting an IVI. Additionally, the release method for convention guide wires could cause significant damage to the semen duct if used with an IVI.

The present invention discloses a new effective and efficient instrument capable of implanting an IVI or similar guide wire based device into the anatomy of a body.

### OBJECTS AND SUMMARY OF THE INVENTION

It is an object of the present invention to provide a cannula implantation instrument capable of introducing an implant in fewer steps than with the conventional designs.

It is another object of the present invention to provide a cannula implantation instrument which minimizes the introduction of germs into a surgical site.

It is yet another object of the present invention to provide a cannula implantation instrument structured to simultaneously introduce a guide wire and cannula into an anatomical cavity or body.

It is a further object of the present invention to provide a cannula implantation instrument structured to minimize the physician's contact with an implantable device during its insertion into an anatomical body.

It is yet a further object of the present invention to provide a cannula implantation instrument ergonomically and compactly structured to contour to a clinician's hand to promote ease of handling.

It is yet a further object of the present invention to provide a cannula implantation instrument which reduces the complexity of the implantation procedure so to allow general urologists to perform the procedure without the assistance or surgical specialists.

It is yet a further object of the present invention to provide a method for inserting an implant into an anatomical portion of a patient.

It is yet a further object of the present invention to provide a method for inserting a cannula and guide wire into an anatomical portion of a patient.

It is still another object of the present invention to provide a cannula implantation instrument and method for the delivery of an intra vas implant or similar devices which overcome the inherent disadvantages of know delivery devices and methods.

In accordance with one form of the invention, a cannula implantation instrument used for implanting a guide wire based implant into an anatomical portion of a patient includes a handle, a cannula and a guide wire, the cannula being mountable to the handle and the handle being capable of receiving the guide wire in a recessed portion thereof. The handle further preferably includes a recessed portion for removably receiving the implant pre-threaded onto the guide wire. The handle may further include a lockable portion located along its body capable of releasably gripping and immobilizing the guide wire during insertion of the cannula into an anatomical portion or body of a patient. The handle may be contoured to ergonomically conform to the natural grip of a physician's hand. During implantation, the cannula and guide wire recessed therein are simultaneously inserted, for example into the vas deferens of a male patient. The lockable portion of the handle is released and the guide wire and implantable device situated thereon become disengaged from the handle. The cannula mounted on the handle is removed from the vas deferens and the guide wire remains. The implantable device, pre-threaded on the guide wire, is advanced over the wire into the vas deferens. The guide wire may then be removed, and the surgical site then sealed.

A preferred form of the cannula implantation instrument and method of using the same, as well as other embodiments, objects, features and advantages of this invention, will be apparent from the following detailed description of illustrative embodiments thereof, which is to be read in conjunction with the accompanying drawings.

### BRIEF DESCRIPTION OF THE DRAWINGS

FIG. 1A is a side view of the cannula implantation instrument formed in accordance with a first embodiment of the present invention.

FIG. 1B is a top view of the cannula implantation instrument formed in accordance with the first embodiment of the present invention.

FIG. 1C is a perspective view of the cannula implantation instrument formed in accordance with the first embodiment of the present invention.

FIG. 1D is an enlarged perspective view of a portion of the cannula implantation instrument formed in accordance with the first embodiment of the present invention.

FIG. 2A is a top view of the cannula implantation instrument formed in accordance with a second embodiment of the present invention.

FIG. 2B is an enlarged top view of a first portion of the cannula implantation instrument formed in accordance with the second embodiment of the present invention.

FIG. 2C is an enlarged top view of a second portion of the cannula implantation instrument formed in accordance with the second embodiment of the present invention.

FIG. 2D is a side view of the cannula implantation instrument formed in accordance with the second embodiment of the present invention, and illustrating the simulated use of the instrument for introducing an implant into an anatomical body.

FIG. 2E is an enlarged side view of a portion of the cannula implantation instrument formed in accordance with the second embodiment of the present invention, and illustrating the simulated use of the instrument for introducing an implant into an anatomical body.

FIG. 2F is a side view of the cannula implantation instrument formed in accordance with the present invention, and illustrating the simulated use of the instrument for introducing an implant into an anatomical body.

FIG. 3A is a top view of the cannula implantation instrument formed in accordance with a third embodiment of the present invention.

FIG. 3B is a perspective view of the cannula implantation instrument formed in accordance with the third embodiment of the present invention.

FIG. 3C is a perspective view of the cannula implantation instrument formed in accordance with a third embodiment of the present invention being held by a physician.

### DETAILED DESCRIPTION OF THE PREFERRED EMBODIMENTS

The present invention is a cannula implantation instrument 2 capable of inserting an intra vas implant 4 (“IVI”) or similar guide wire based device into an anatomical portion or body of a patient. The method and apparatus of the present invention provide a fast, efficient, safe and non-traumatic method for introducing an IVI 4 or similar device into the anatomical portion of a patient, and more specifically into the vas deferens of a male patient. The cannula implantation instrument 2 of the present invention simplifies the implantation procedure for the physician and subsequently requires a practitioner less specialized in the surgical field than that required when using conventional apparatus and methods.

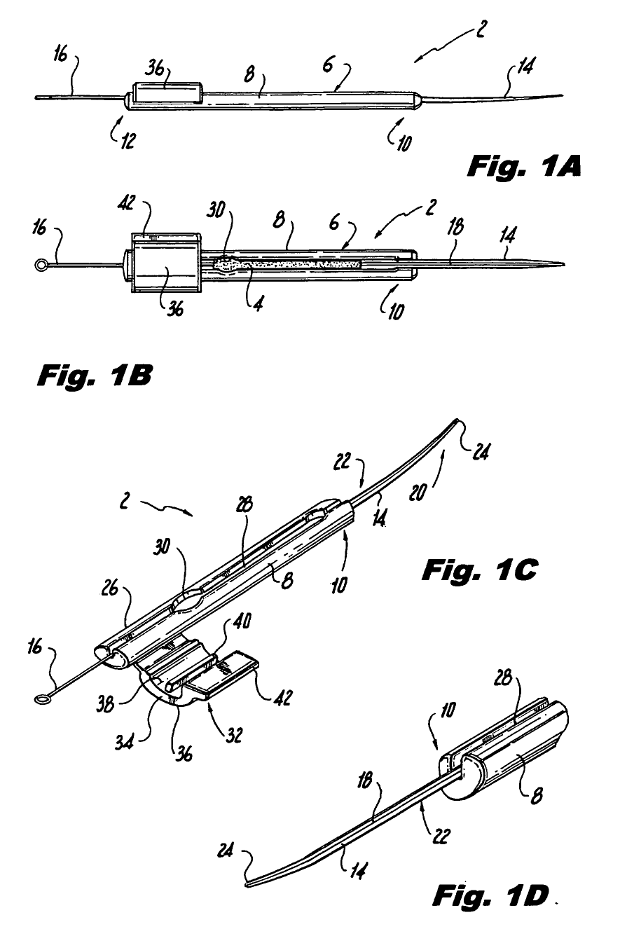

Initially referring to FIGS. 1A-1D of the drawings, in a first embodiment, the cannula implantation instrument 2 of the present invention includes a handle 6 in the form of an elongated member 8 having a first axial end 10 and a second axial end 12 situated opposite the first axial end 10, a cannula 14 mounted on the first axial end 10 of the handle 6, and a guide wire 16. The cannula 14 is preferably folded along its longitudinal length to define an open groove 18 or slot capable of removably receiving the guide wire 16. The cannula 14 includes a first axial end 20 and a second axial end 22 situated opposite the first end 20. The first axial end 20 may be slightly curved and is formed with a sharpened tip 24, and the second axial end 22 may be molded or otherwise mounted on the handle 6 at the first axial end 10 of the handle 6. The elongated handle 6 is preferably constructed of a medical grade polymer, such as polyurethane or polyethylene, and may be formed by injection molding or similar method.

The handle 6 has an outer surface 26 in which is formed an open slot 28 extending longitudinally along at least a portion of the length of the handle 6. The slot 28 formed in the handle surface 26 is aligned with the open slot 18 formed in the cannula 14 so that the slots 28,18 are in mutual communication to removably receive the guide wire 16 extending through the handle slot 28 and cannula slot 18. The handle 6 further has formed in its outer surface 26 a recessed open cavity 30 which is in communication with the guide wire slot 28 formed therein. The cavity 30 is formed with a complementary shape to that of the implant 4 so that the implant 4 may be removably received by the cavity 30. As mentioned previously, the implant 4 is pre-threaded, or beaded, on the guide wire 16. The communication between the slot 28 in the handle 6 and the cavity 30 allows the pre-threaded implant 4 and guide wire 16 to be removably mounted on the handle 6 in one piece.

The handle 6 further includes a clip 32 hingedly mounted on the surface 26 of the handle 6 to straddle the slot 28 and/or cavity 30 formed in the handle 6. More specifically, the clip 32 is mounted to the outer surface 26 of the handle 6 by way of a living hinge. The clip 32 has a main body 34 having an outer surface 36 and an inner surface 38 opposite the outer surface 36. The main body 34 of the clip 32 also preferably has a rib 40 extending outwardly from the inner surface 38 thereof. The rib 40 extends in the same general direction as the handle slot 28 so as to be received by the slot (or cavity) to retain the guide wire 16 in the slot 28 or the implant 4 within the cavity 30 when the clip 32 is in the closed position. The clip 32 is preferably complementary shaped to that of the outer surface 26 of the handle 6 so that the inner surface 38 of the clip 32 can engage or at least be in close proximity to the outer surface 26 of the handle 6 when the clip 32 is in a closed position straddling the slot 28 or cavity 30 formed in the handle 6.

The clip 32 further preferably includes an extended portion 42 mounted on the main body 34 and extending outwardly therefrom so that the physician may engage the extended portion 42 with his or her finger to pivot the clip 32 from the closed position, in which the clip 32 straddles the slot 28 or cavity 30, to an open position, in which the clip 32 uncovers the slot 28 or cavity 30 to allow the guide wire 16 and implant 4 to be removed respectively therefrom.

The clip 32 is also preferably formed to be partially or slightly resilient, as required, to engage at its extended portion 42 the outer surface 26 of the handle 6 to releasably secure the clip 32 in the closed position. A small protrusion (not shown) extending outwardly from the outer surface 36 of the clip 32 may be provided to engage the extended portion 42 of the clip 32 to releasably secure the clip 32 in the closed position. Or, even more preferably, the handle 6 may be shaped to be generally oval in transverse cross-section, with the clip 32 similarly shaped, so that the resilient clip 32 at the extended portion thereof may engage and partially overlap a portion of the outer surface intersecting at the major axis of its oval cross-sectional shape to retain the clip 32 in the closed position until it is forcibly released from engagement with the handle outer surface 26 by the physician.

Thus, the guide wire 16 and implantable device 4, once threaded together, may be received and housed by the slot 28 and cavity 30 in the handle 6 and the aligned slot 18 of the cannula 14. The securing clip 32 may be hingedly closed over the handle 6, engaging both the handle 6 and guide wire 16, or the implantable device 4, preventing any axial or radial movement of the wire 16 or the implantable device 4 within the handle 6.

During insertion, the sharpened tip 24 of the cannula 14 pierces the vas deferens of the patient, and the cannula 14 and guide wire 16 contained therein may be inserted simultaneously into the pierced vas deferens. The securing clip 32 is released, disengaging the guide wire 16 and threaded implantable device 4 from the handle 6. The cannula 14 and handle 6 may then be retracted from the vas deferens while an end portion of the guide wire 16 remains within the vas deferens. The threaded implantable device 4 may then be axially moved along the guide wire 16 into the vas deferens of the patient to a desired position therein. Once inserted, the guide wire 16 can be retracted and the surgical site sealed.

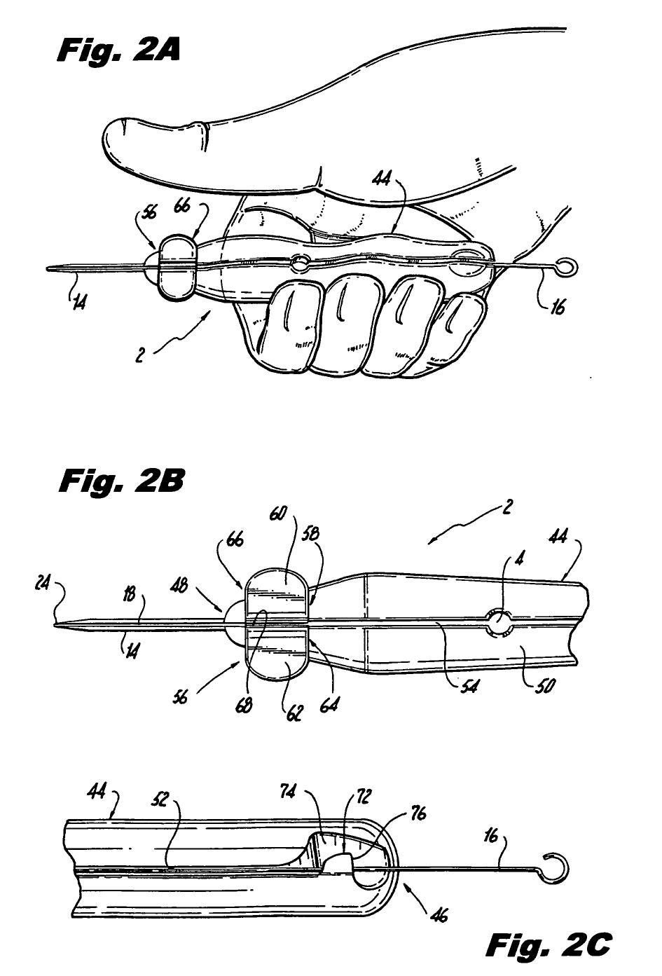

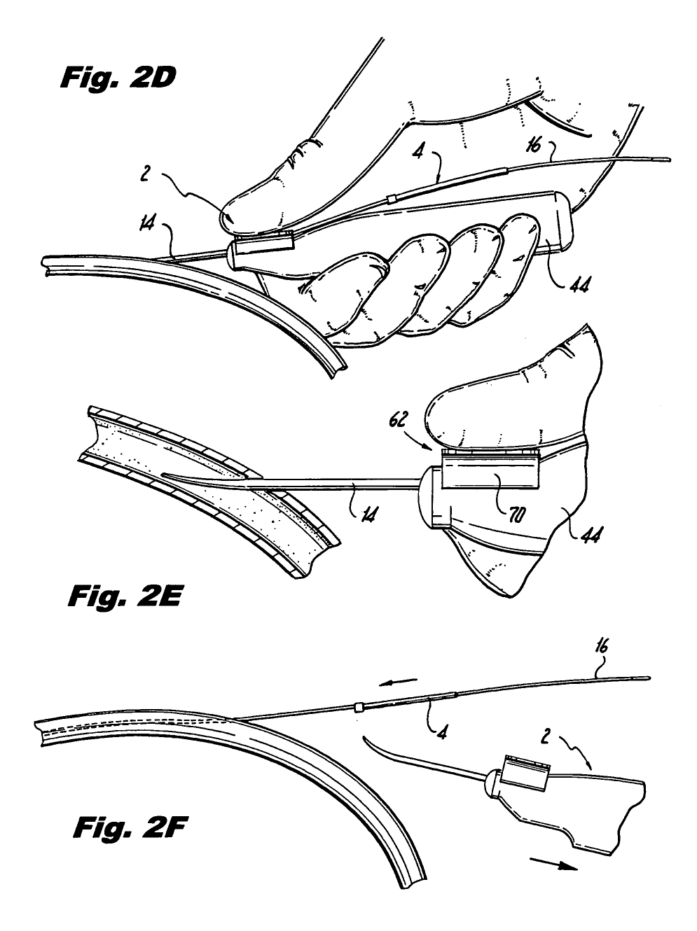

Now referencing FIGS. 2A-2G of the drawings, in accordance with a second embodiment of the present invention, the cannula implantation instrument 2 includes a handle 44 that is constructed to contour more closely to the physician's hand, allowing better handling and maneuverability. As in the first embodiment shown in FIGS. 1A-1D and described previously, the contoured handle 44 of the second embodiment includes a first axial end 46 and an opposite second axial end 48, and has formed in the outer surface 50 thereof a slot 52 and interconnected cavity 54 for respectively removably receiving therein the guide wire 16 and implant 4 pre-threaded on the guide wire 16. As in the previously described embodiment, the present embodiment shown in FIGS. 2A-2F includes a cannula 14 having folded over sides to define an open slot 18 for receiving an end portion of the guide wire 16, which slot 18 is in alignment and communication with the open slot 52 and open cavity 54 of the handle 44 for respectively receiving the guide wire 16 and the implant 4 mounted on the guide wire 16. The cannula 14 has a first axial end 20, which is mounted on the second axial end 48 of the handle 44, and has a second axial end 22 which is opposite the first axial end 20 and which is formed with a sharpened tip 24.

The second embodiment of the cannula implantation instrument 2 has a preferred securing clip 56 which is different in structure and operation from the securing clip 32 of the first embodiment described previously. As shown in FIGS. 2A-2F, the securing clip 56 of the second embodiment is in the form of two bent resilient members 58. More specifically, the locking or securing clip 56 may include a first elongated, plate-like member 60, and a second elongated, plate-like member 62. Each of the first and second members 60,62 has a first axial end 64 and a second axial end 66 situated longitudinally opposite the first axial end 64. The two first axial ends 64 of the members 60,62 are joined together, with the members extending from this juncture in generally the same direction. Each clip member 60,62 includes opposite inner and outer surfaces 68,70, with the inner surfaces 68 defining clamping surfaces for clamping the guide wire 16 therebetween. The inner or clamping surface 68 of the first member 60 is disposed to face the inner or clamping surface 68 of the second member 62, with little space between the clamping surfaces of the clip members near where they are joined at their first axial ends 64. Thus, the two clip members 60,62 define two clamping jaws so that the guide wire 16 of the implantation instrument 2 may be releasably clamped by and between the two members and in particular the clamping surfaces thereof near where the first axial ends of each are joined. The joined first axial ends of the first and second members 60,62 are mounted to the handle 44 and are preferably positioned within the open slot 52 of the handle 44 so that they may receive between them the guide wire 16 that resides in the slots 52, 18 of the handle and cannula.

The second ends 66 of the first and second clip members 60,62 extend away from the outer surface 50 of the handle 44 so as to be accessible by the physician during the implantation procedure. The second ends 66 of the first and second clip members 60,62 are also outwardly flared or bent so that they mutually diverge. More specifically, the second ends 66 of the clip members 60,62 reside outside the handle slot 52 and are exposed for the physician to rest his finger thereon and exert pressure thereon to separate the two clip members 60,62 and release the guide wire 16 wedged therebetween.

The clip 56 could be formed from a resilient material, such as spring stainless steel, by folding in half an elongated piece of such material to define the first and second clip members 60,62, and with the juncture of the first axial ends 64 of the clip members 60,62 residing at the fold in the material. Alternatively, the clip 56 may be molded from a polymer material.

The clip 56 is preferably situated near the second axial end 48 of the handle 44, in proximity to where the physician's fingers would be located when grasping the handle 44, so that the implantation instrument 2 may be maneuvered, and the guide wire 16 and pre-threaded implant 4 released therefrom, by the physician using one hand.

The second embodiment of the cannula implantation instrument 2 further includes a hook 72, as shown by FIG. 2C of the drawings, which is preferably situated near the first axial end 46 of the handle 44 opposite the second axial end 48 on which the cannula 14 is mounted. More specifically, a portion of the outer surface 50 of the handle 44 defining a wall of the slot 52 is angularly recessed to form the slot wall portion with a beveled or sloped surface 74. Situated partially over the slot 52 and sloped portion 74 of the outer surface 50 is an overhanging portion 76 of the outer surface 50. The overhanging portion 76 defines the hook 72 which maintains the guide wire 16 within the handle slot 52 at the first axial end 46 thereof. To remove the guide wire 16 having the pre-threaded implant 4 situated thereon from the slot 52 of the handle 44, the physician presses down on the flared second ends 66 of the clip members 60,62 to unclamp the guide wire 16 at the second axial end 48 of the handle 44, and maneuvers the guide wire 16 laterally with respect to the handle 44 at the first axial end 46 thereof between the sloped portion 74 and the overhanging portion 76 of the outer surface 50. Now, the guide wire 16 and implant 4 mounted thereon are free of the handle 44 and cannula 14. Of course, it should be understood that the physician may first release the guide wire 16 from the hook 72 at the first axial end 46 of the handle 44, and then release the guide wire 16 at the second axial end 48 of the handle 44 by pressing down with his finger on the flared second ends 66 of the clip members 60,62. The cannula 14 is then withdrawn from the vas deferens while the end portion of the guide wire 16 remains within the vas deferens, allowing the implantable device 4 situated thereon to be guided into the vas deferens. The guide wire 16 is retracted once the implantable device 4 is properly positioned within the vas deferens, and the surgical site is sealed.

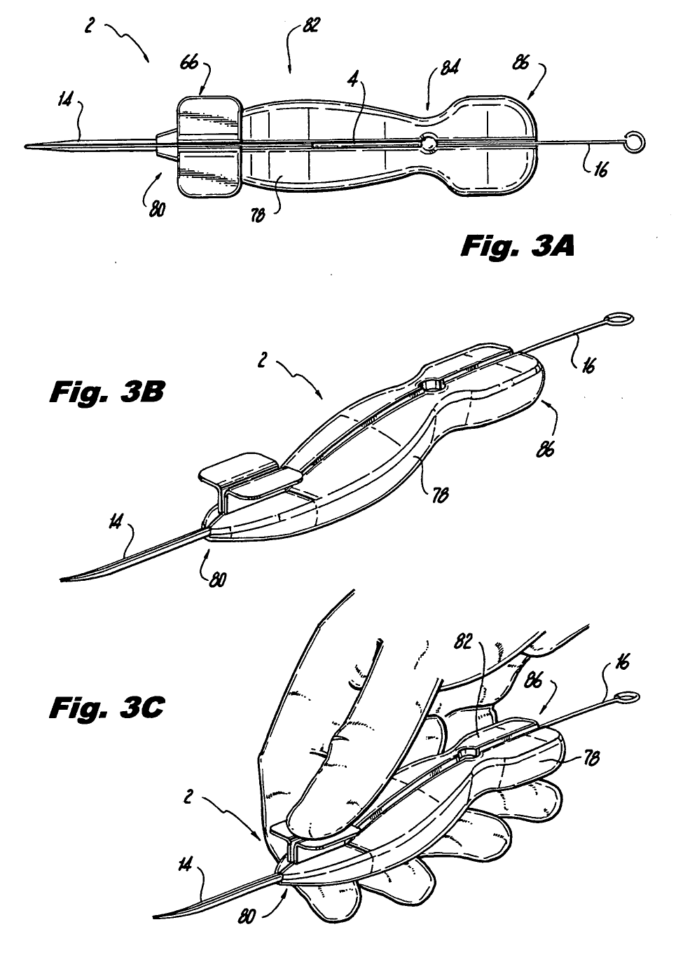

A third embodiment of a cannula implantation instrument 2 formed in accordance with the present invention is shown in FIGS. 3A-3C. The structure and operation of this embodiment is similar to those of the second embodiment described previously and shown in FIGS. 2A-2F. In this third embodiment, however, the handle 78 is longitudinally and laterally shaped for even better and more comfortable handling by the physician. The handle 78 has a generally hour-glass shape, with a front portion 80 on which the cannula 14 is mounted, a main body portion 82 situated longitudinally adjacent the front portion 80, a reduced diameter portion 84 situated longitudinally adjacent the main body portion 82, and a slightly bulbous rear portion 86 situated longitudinally adjacent the reduced diameter portion 84 and opposite the front portion 80 of the handle 78 on which the cannula 14 is mounted. Also, the cannula 14 may be removed from the handle 78 and replaced with a different cannula having, for example, a shorter or longer length, or a different shape or curvature, to meet the requirements of the physician. Furthermore, certain portions of the handle 78 are offset or angled longitudinally from each other. For example, the front portion 80 and bulbous rear portion 86 of the handle 78 of this embodiment may reside generally in different, substantially parallel planes, while the main body portion 82 is angled to and situated between the front and rear portions 80,86.

The present invention overcomes the disadvantages inherent in the conventional implantation devices by simplifying the structure of such devices and their use in performing an implantation procedure. Utilization of the present invention may reduce the possibility of infection by limiting the amount of contact the physician has to make with the implantable device. The difficulty of use associated with the conventional designs, such as beading the implant, is eliminated, and the placement of the implant can be done by a general urologist, without requiring the specialized skills of a surgeon.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

What is claimed is:

1. An implantation instrument for use with guide wire based implants, which comprises:
an elongated handle having an axial length, the handle having a first axial end and a second axial end situated opposite the first axial end, and an outer surface, the outer surface having formed therein an open slot extending at least partially along the axial length thereof for removably receiving a first portion of a guide wire; and
a cannula, the cannula being mounted on the handle at the second axial end thereof, the cannula having an outer surface in which is formed an open slot extending at least partially along the length thereof for removably receiving a second portion of the guide wire, the slot of the cannula being in communication with the slot of the handle to allow the guide wire to extend between the handle and the cannula.

2. An implantation instrument as defined by claim 1, wherein the outer surface of the handle has further formed therein an open cavity for removably receiving the implant, the open cavity being in communication with the slot of the handle and the slot of the cannula.

3. An implantation instrument as defined by claim 1, which further comprises:
a locking clip, the locking clip being hingedly joined to the outer surface of the handle, and being positionable thereon in at least a first position, wherein the clip covers a portion of the open slot formed in the handle to retain a guide wire within the open slot of the handle, and a second position, wherein the clip uncovers the portion of the open slot of the handle to allow a guide wire received by the open slot of the handle to be removed therefrom.

4. An implantation instrument as defined by claim 3, wherein the locking clip includes an inner surface, and a rib extending outwardly from the inner surface, the rib being at least partially received in the open slot of the handle to further retain a guide wire within the open slot of the handle when the locking clip is in the first position.

5. An implantation instrument as defined by claim 4, wherein the rib extends sufficiently into the open slot of the handle to be engageable with a guide wire received by the open slot in the handle to minimize movement of the guide wire in an axial direction and in a radial direction within the open slot of the handle when the locking clip is in the first position.

6. An implantation instrument as defined by claim 1, which further comprises:
a locking clip, the locking clip being mounted on the handle and releasably engageable with a guide wire received by the open slot of the handle.

7. An implantation instrument as defined by claim 6, wherein the locking clip includes a first clip member and a second clip member, each of the first and second clip members having a first axial end and a second axial end situated opposite the first axial end, and a clamping surface for releasably engaging a guide wire received by the open slot of the handle, the clamping surfaces of the first and second clip members facing each other, the second ends of the first and second members extending outwardly from the outer surface of the handle to be engageable by a user of the implantation instrument, whereby engagement of the second ends of the first and second clip members by the user of the implantation instrument causes the clamping surfaces of the first and second members to separate from each other to allow a guide wire positioned therebetween to be removed from between the clamping surfaces.

8. An implantation instrument as defined by claim 1, which further comprises:
a hook structure, the hook structure being formed on the outer surface of the handle and partially overhanging a portion of the open slot of the handle to help selectively retain the guide wire within the open slot of the handle.

9. An implantation instrument as defined by claim 8, wherein the hook structure includes a portion of the outer surface of the handle which extends over a portion of the open slot formed in the handle.

10. An implantation instrument as defined by claim 1, wherein the second axial end of the cannula includes a sharpened tip.

11. A method of implanting a guide wire based implant in a patient utilizing a cannula implantation instrument as defined by claim 2, which comprises the steps of:
introducing the second axial end of the cannula of the implantation instrument into an anatomical portion of the patient, the implantation instrument having mounted thereon a guide wire received by the open slot of the handle and the open slot of the cannula and the implant mounted on the guide wire and received by the open cavity of the handle;
withdrawing the second axial end of the cannula from the anatomical portion of the patient with at least a portion of the guide wire remaining in the anatomical portion of the patient;
advancing the implant over the guide wire and into the anatomical portion of the patient to a desired position therein; and
removing the guide wire from the anatomical portion of the patient.

12. An implantation instrument for use with guide wire based implants, which comprises:
an elongated handle having an axial length, the handle having a first axial end and a second axial end situated opposite the first axial end, and an outer surface, the outer surface having formed therein an open slot extending at least partially along the axial length thereof for removably receiving a first portion of a guide wire, and having further formed therein an open cavity for removably receiving the implant which is mounted on the guide wire, the open cavity being in communication with the open slot of the handle;
a cannula, the cannula having a first axial end and a second axial end situated opposite the first axial end, the first axial end of the cannula being mounted on the second axial end of the handle, the second axial end of the cannula being formed with a sharpened tip, the cannula having an outer surface defining an open slot extending at least partially along the length thereof for removably receiving a second portion of the guide wire, the slot of the cannula being in communication and alignment with the slot of the handle to allow the guide wire to extend between the handle and the cannula; and
a locking clip, the locking clip being situated on the handle and being engageable by a user of the implantation instrument, and further being in at least a first state, wherein the locking clip retains the guide wire within the open slot of the handle, and a second state, wherein the locking clip permits the guide wire to be removed from the open slot of the handle.
